(19) United States
(12) Patent Application Publication
(10) Pub. No.: US 2002/0009807 A1
(43) Pub. Date: Jan. 24, 2002

(54) Lipid bilayer array method and devices

(76) Inventors: Lance Kam, Palo Alto, CA (US); Steven G. Boxer, Stanford, CA (US)

Correspondence Address: IOTA PI LAW GROUP, 350 CAMBRIDGE AVENUE SUITE 250, P O BOX 60850, PALO ALTO, CA, 94306-0850, US

(21) Appl. No.: 09/860,124

(22) Filed: May 17, 2001

Related U.S. Application Data
(60) Provisional application No. 60/205,604, filed on May 18, 2000.

(51) Int. Cl. C12N 5/00

(52) U.S. Cl. 435/402

(57) ABSTRACT

The invention provides useful devices and methods for both studying interfaces between cell membranes, and integrating living cells with synthetic surfaces exhibiting complex lateral composition, organization and fluidity. Described is the fabrication of controlled interfaces between cells and synthetic supported lipid bilayer membranes.

This application claims the benefit of U.S. Provisional Application No. 60/205,604 filed May 18, 2000, which is incorporated herein by reference in its entirety.

### FIELD OF THE INVENTION

This invention relates to the fields of cell culture, cell physiology, lipid bilayers, cell adhesion, microcontact printing, micropatterning, and endothelial cells.

### REFERENCES

1. Sackmann, E., Supported membranes: scientific and practical applications. Science 271:43-48, (1996).

2. McConnell, H. M., et al., Supported planar membranes in studies of cell-cell recognition in the immune system. Biochim. Biophys. Acta 864:95-106, (1986).

3. Johnson, S. J., et al., Structure of an adsorbed dimyristoylphosphatidylcholine bilayer measured with specular reflection of neutrons. Biophys. J. 59:289-294, (1991) .

4. Koenig, B. W., et al., Neutron reflectivity and atomic force microscopy studies of a lipid bilayer in water adsorbed to the surface of a silicon single crystal. Langmuir 12:1343-1350, (1996).

5. Giancotti, F. G. and Ruoslahti, E., Transduction Integrin signaling. Science 285:1028-1032, (1999).

6. Viola, A. and Lanzavecchia, A., T-cell activation and the dynamic world of rafts. Apmis 107:615-623, (1999).

7. Watts, T. H., and McConnell, H. M., Biophysical aspects of antigen recognition by T cells. Ann. Rev. Immun. 5:461-475, (1987).

8. Grakoui, A., et al., The immunological synapse: a molecular machine controlling T cell activation. Science 285:221-227, (1999).

9. Margolis, L. B., Cell interaction with model membranes. Probing, modification and simulation of cell surface functions. Biochim. Biophys. Acta 779:161-189, (1984).

10. van Oudenaarden, A. and Boxer, S. G., Brownian ratchets: Molecular separations in lipid bilayers supported on pattered arrays. Science 285:1046-1048, (1999).

11. Groves, J. T., et al., Micropatterning of fluid lipid bilayers on solid supports. Science 275:651-653, (1997).

12. Groves, J. T. and Boxer, S. G., Electric field induced concentration gradients in planar supported bilayers. Biophys. J. 69:1972-1975, (1995).

13. Hovis, J. S. and Boxer, S. G., Patterning barriers to lateral diffusion in supported lipid bilayer membranes by blotting and stamping. Langmuir 16:894-897, (2000).

14. Hui, S. W., et al., The structure and stability of phospholipid bilayers by atomic force microscopy. Biophys. J. 68:171-178, (1995).

15. Radler, J., et al., Velocity-dependent forces in atomic-force microscopy imaging of lipid films. Langmuir 10:3111-3115, (1994).

16. Kumar, A., et al., Patterning self-assembled monolayers: applications in material science. Langmuir 10:1498-1511, (1994).

17. Bernard, A., et al., Printing patterns of proteins. Langmuir 14:2225-2229, (1998).

18. Kam, L., et al., Neuron attachment and outgrowth on microcontact-printed polylysine-conjugated laminin. J. Neurosci. Meth. (in press).

19. Chen, C. S., et al., Geometric control of cell life and death. Science 276:1425-1428, (1997).

20. Cremer, P. S. and Yang, T., Creating spatially addressed arrays of planar supported fluid phospholipid membranes. J. Am. Chem. Soc. 121:8130-8131, (1999).

21. Kung, L. A., et al., Printing via photolithography on micropartitioned fluid lipid membranes. Adv. Mat. (in press).

22. Saxon, E. and Bertozzi, C. R., Cell surface engineering by a modified Staudinger reaction. Science 287:2007-2010, (2000).

23. Lin-Liu, S., et al., Migration of cell surface concanavalin A receptors in pulsed electric fields. Biophys. J. 45:1211-1217, (1984).

24. Webb, W. W., et al., Molecular mobility on the cell surface. Biochem. Soc. Symp. 191-205, (1981).

### BACKGROUND OF THE INVENTION

Supported lipid bilayers mimic many features of cell membranes and are useful for interfacing living cells with synthetic surfaces, for studies of complex interactions between membrane surface components, and for applications such as implant biomaterials and biosensors (see Ref. 1, incorporated by reference herein). Supported lipid bilayers consist of two opposed phospholipid leaflets in close association with an appropriate hydrophilic surface such as glass (see Ref. 2, incorporated by reference herein). A layer of water several nanometers thick separates the membrane from the support (see Ref. 3 and Ref. 4, both incorporated by reference herein). Consequently, molecular components in lipid bilayers of appropriate composition freely diffuse within the plane of the membrane, mimicking a property of cellular membranes that is essential for many cell functions (see Ref. 5 and Ref. 6, both incorporated by reference herein). Furthermore, the composition and fluid properties of supported lipid bilayers are easily controlled, providing a robust tool for the study of numerous systems ranging from integral membrane proteins (e.g., integrins, gap junctions, and GPI-anchored proteins) to cells of the immune system (see Ref. 1, Ref. 2, Ref. 6, Ref. 7, and Ref. 8, each incorporated by reference herein).

A fundamental issue facing the use of supported lipid bilayers with anchorage-dependent cells is that these cells cannot form stable attachments with fluid lipid structures (see Ref. 9, incorporated by reference herein).

### SUMMARY OF THE INVENTION

The invention provides methods for micropatterning lipid bilayers resulting in devices that facilitate adhesion of anchorage-dependent cells onto fluid membranes by the microfabrication of regions that direct and corral lipid diffusion on surfaces from materials such as TiOₓ and photoresist as described by Ref. 10 and Ref. 11, and as described in copending U.S. patent application Ser. No. 09/680,637, filed Oct. 6, 2000, and U.S. Pat. No. 6,228,326, filed Nov. 26, 1997, all of which are incorporated by reference herein, or by selectively removing regions of the assembled bilayer, as described by Ref. 10 and Ref. 11, both incorporated by reference herein. Micropatterned bovine serum albumin (BSA), for example, can be used as a barrier to pattern bilayers. Printing barriers of biologically-active molecules such as BSA imparts additional functionality to micropatterned lipid bilayers.

The invention further provides a surface detector array device for adhering cells over lipid bilayer expanses. The device comprises a substrate having a surface defining a plurality of distinct bilayer-compatible surface regions separated by one or more bilayer barrier regions. Furthermore, the bilayer-compatible surface regions and the bilayer barrier surface regions are formed of different materials, and the bilayer barrier regions further include a cell adhesion compatible material. Lipid bilayer expanses are stably localized on each of the bilayer-compatible surface regions such that an aqueous film is interposed between each bilayer-compatible surface region and corresponding lipid bilayer expanse. Also, each lipid bilayer expanse is stably localized above each bilayer-compatible surface in the absence of covalent linkages between each lipid bilayer expanse and each bilayer-compatible surface, and separated therefrom by said aqueous film. A bulk aqueous phase covers the lipid bilayer expanses.

The invention further provides a method for adhering cells to a surface array of lipid bilayer expanses. The method comprises the steps of (1) providing a surface, and (2) creating lipid bilayer compatible regions surrounded by bilayer barrier regions on the surface. The bilayer barrier regions further comprise a cell adhesion compatible material. (3) Covering the surface with a bulk aqueous phase, and (4) forming one or more lipid bilayer expanses above the lipid bilayer compatible regions. The lipid bilayer expanse is stably localized above the bilayer-compatible surface in the absence of covalent linkages between each lipid bilayer expanse and each bilayer-compatible surface, and separated therefrom by an aqueous film formed from a portion of the bulk aqueous phase. (5) adhering cells to the cell adhesion compatible material such that the cells adhere only to the cell adhesion compatible material and not to the lipid bilayer expanse.

### BRIEF DESCRIPTION OF THE FIGURES

FIGS. 1A-1D depict micropatterning of substrates with fibronectin and phospholipid bilayers.

FIGS. 2A-2C show that fluid lipid bilayers do not support endothelial cell adhesion.

FIGS. 3A-3C depict adhesion of endothelial cells onto surfaces modified with squares of fibronectin.

FIGS. 4A-4B depict adhesion of endothelial cells onto surfaces modified with grids of fibronectin.

FIGS. 5A-5B depict lipid bilayers underlying adherent cells remain fluid.

FIG. 6 depicts a cell on a surface having bilayer compatible regions and bilayer barrier regions.

### DETAILED DESCRIPTION OF THE FIGURES

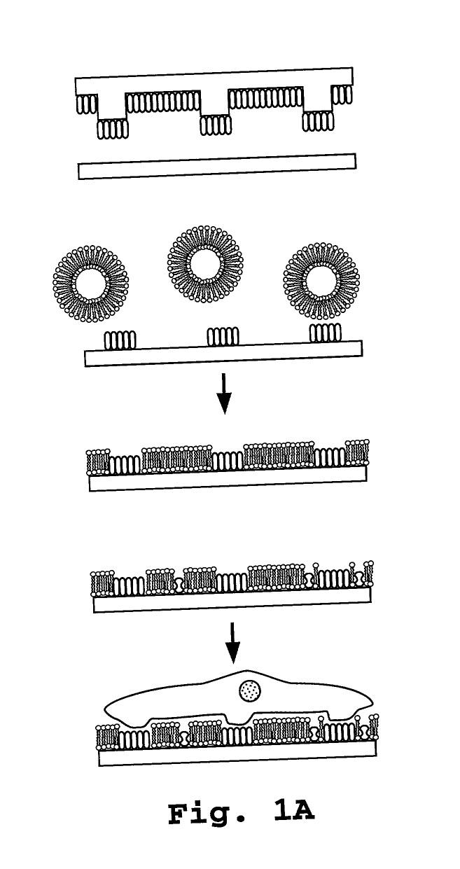

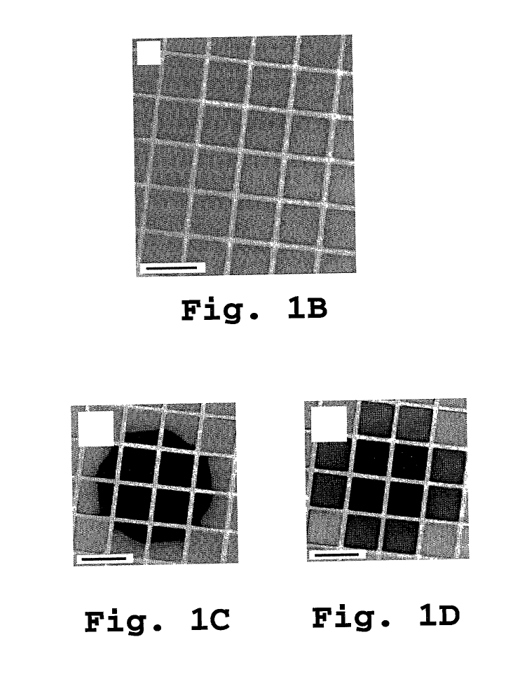

FIG. 1A depicts micropatterned supported lipid bilayer membranes by using the cell adhesive protein fibronectin. This not only patterns and corrals the supported bilayers, but also provides stable anchorages for cells, thereby promoting and directing the interaction between the cells and supported membranes.

Micropatterning of substrates with fibronectin and phospholipid bilayers. FIG. 1A depicts a schematic outlining the process used to create protein-micropatterned lipid bilayer surfaces. First, barriers of fibronectin were microcontact printed onto glass. These barriers limited the fusion of SUVs (small unilamellar vesicles)into lipid bilayers onto only the regions of the substrate not covered by fibronectin. FIG. 1B shows a surface containing gridlines of fibronectin measuring 5 μm in width and spaced 40 μm apart; fluorescently-labeled lipid bilayers in the corrals formed by these barriers are shown. FIG. 1C is an image of an octagonal pattern photobleached onto an array of 16 lipid corrals. This image was taken immediately after photobleaching, illustrating the different fractions of NBD-PE in adjacent corrals that underwent photodamage. FIG. 1D is an image ten minutes later showing the lipids within each corral mixed completely, demonstrating both that the lipid bilayers were fluid and that neighboring corrals were isolated from each other. The scale bar in each image is 50 μm.

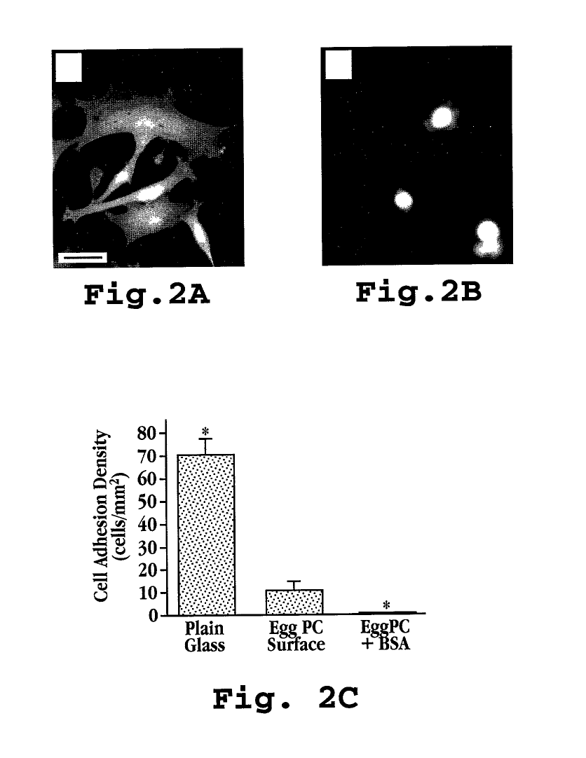

FIGS. 2A-2C show that the fluid lipid bilayers do not support endothelial cell adhesion. FIG. 2A is an image taken after 6 hours in serum-free media showing endothelial cells on substrates of plain glass exhibit a well spread morphology. In contrast, FIG. 2B shows cells on surfaces supporting a fluid lipid bilayer of egg phosphatidylcholine exhibit a rounded morphology. Cell adhesion is reduced on lipid bilayers (egg PC) compared to plain glass as shown by the first and second entries in FIG. 2C. Cell adhesion is further reduced by passivating the supported bilayers with 10 mg/ml of bovine serum albumin (egg PC+BSA as shown in FIG. 2C). Cells on egg PC+BSA surfaces resembled that on egg PC alone (FIG. 2B). FIGS. 2A and 2B are presented at identical magnification; the scale bar in FIG. 2A is 25 μm. The data in FIG. 2C are mean ±S.E.M., n=3. *P<0.005 (least significant difference test) compared to the substrate of egg PC.

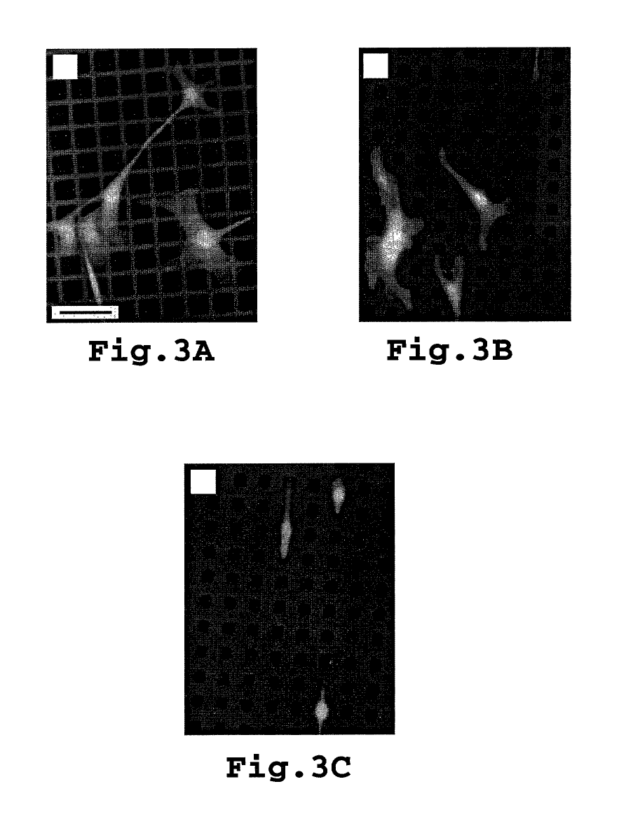

FIGS. 3A-3C depict adhesion of endothelial cells onto surfaces modified with squares of fibronectin. Six-hour adhesion of endothelial cells, under serum-free conditions, onto surfaces patterned with (dark) square features of fibronectin surrounded by supported bilayers of egg PC/NBD-PE . Cells were labeled with CellTracker Blue. All images are presented at identical magnification; the scale bar in FIG. 3C is 50 μm. The width and spacing of squares in each Figure are as follows: FIG. 3A is 20 μm squares spaced 5 μm apart; FIG. 3B is 10 μm squares spaced 10 μm apart, FIG. 3C is 10 pm squares spaced 30 μm apart.

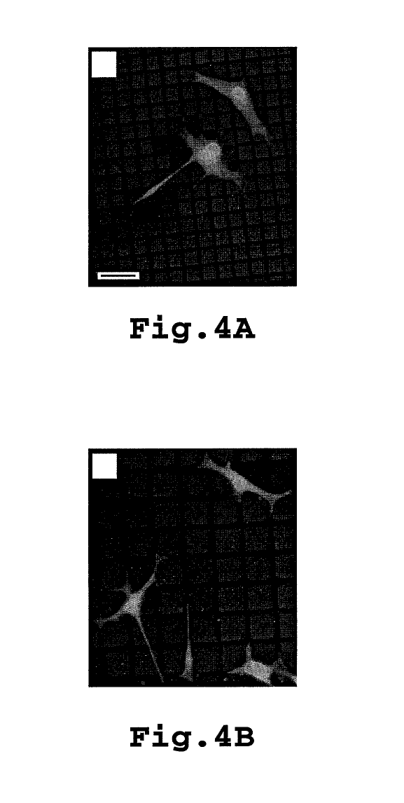

FIGS. 4A-4B depict adhesion of endothelial cells onto surfaces modified with grids of fibronectin. Endothelial cell adhesion onto surfaces patterned with grid-like features of fibronectin (dark horizontal and vertical lines) corralling supported bilayers of egg PC/ NBD-PE. The lipid corrals in each frame measure either 20 μm (separated by 5 μm) for FIG. 4A or 40 μm (separated by 10 μm) in width for FIG. 4B. Cell morphology was independent of the width of the fibronectin grid lines. The scale bar in FIG. 4A is 50 μm.

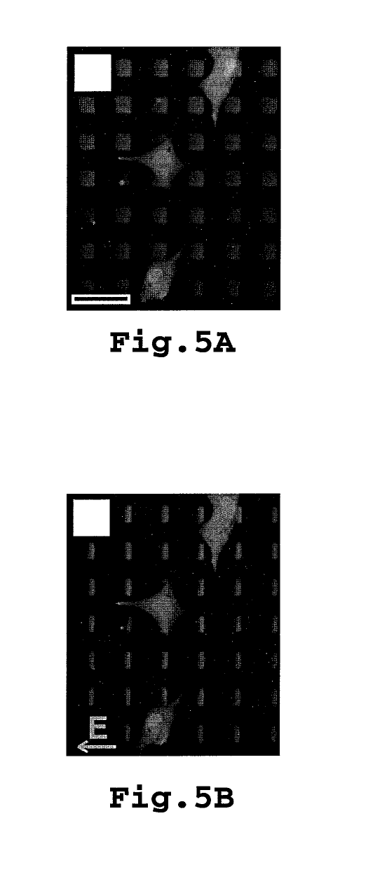

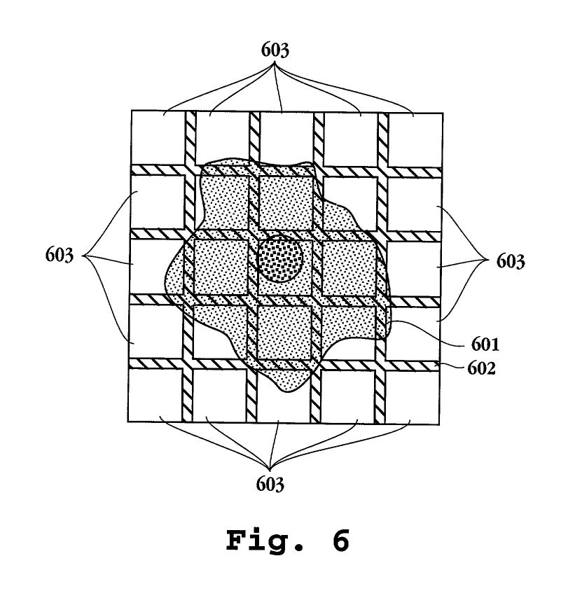

FIG. 5A-5B depict lipid bilayers underlying adherent cells remain fluid. FIG. 5A is an image of endothelial cell adhesion on a surface containing 20-μm-wide corrals containing bilayers of egg PC/TR-PE . FIG. 5B is an image taken after 5 minutes of exposure to a 60 V/cm electric field applied parallel to the membrane surface, the negatively charged TR-PE lipids underlying adherent cells migrated to the right side of each corral identically as those in regions distant from the cells. The scale bar in FIG. 5A is 50 μm.

FIG. 6 represents a cell 601 attached to bilayer barrier regions 602 and spanning bilayer compatible regions 603 with lipid bilayer expanses (not shown) contained within.

### DETAILED DESCRIPTION OF THE INVENTION

The present invention provides methods and devices for bringing anchorage-dependent cells into close proximity with synthetic lipid bilayers with fine topological control. Patterning of either square or grid-like barrier regions of fibronectin onto a lipid bilayer is effective in promoting cell adhesion. These two strategies result in qualitatively different cell-substrate interactions, which provide valuable tools for studying how anchorage-dependent cells recognize and respond to components of cellular membranes. On surfaces containing squares of fibronectin, the complementary regions of lipid bilayer form a single, connected membrane. These canals of fluid lipid bilayer could be used to introduce membrane-incorporated biomolecules into the interface between an adherent cell and the substrate, for example by application of an electric field, as we have shown in a different context as shown in Ref. 10, entirely incorporated by reference herein. By comparison, surfaces modified with grids of fibronectin contain multiple, isolated corrals of lipid. Using recently developed methods for controlling the composition of individual bilayer patches as described in Ref. 13, Ref. 20, and Ref. 21, each of which are entirely incorporated by reference herein, these fibronectin grids should make possible the quantitative study of receptor-specific interactions. By combining canals and corrals of lipids we take a step towards mimicking aspects of the environment encountered by populations of cells organized in tissues. Reorganization of either endogenous or engineered molecular species, as described in Ref. 22, entirely incorporated by reference herein, in membranes of adherent cells using electric fields, a concept that has already proven useful in several contexts as described by Ref. 22 and Ref. 23, may provide additional insight into the mechanisms that regulate cell-membrane interactions. Finally, incorporation of cell-cell communication proteins, such as gap junctions, and electronics integrated into the solid support could be used to probe the internal state of a cell, leading to advanced, cell-based devices.

### Cells do not adhere to passivated supported bilayers

FIGS. 2A-2B compare endothelial cell adhesion on bare glass and on supported lipid bilayers. The presence of a fluid bilayer of egg phosphatidylcholine greatly reduces both the adhesion density and the spreading of cells relative to glass (FIGS. 2A and 2B). Cell adhesion density was further reduced by incubating the supported lipid bilayers with bovine serum albumin (BSA) prior to introduction of cells (FIG. 2C). This passivation step does not disrupt the supported bilayer; the diffusion coefficient of NBD-labeled lipids in unpatterned egg PC bilayers was unaffected by incubation with BSA (1.3±0.5 μm²/sec vs. 1.9±0.9 μm²/sec for BSA incubated and untreated bilayers, respectively; P<0.05). Passivation of lipid bilayers with BSA likely occurs by filling in of defects that are present in supported bilayers (See Ref. 14 and Ref. 15, both entirely incorporated by reference herein).

### Barrier regions effectively direct lipid lateral organization and diffusion

Protein-micropatterned lipid bilayer surfaces are prepared by first patterning glass substrates with fibronectin using microcontact printing as described by Ref. 18 and Ref. 19, both herein incorporated by reference (see FIG. 1A). These surface-bound proteins prevent the fusion of small unilateral vesicles (SUVs) of phosphatidylcholine with the underlying substrate, directing the formation of lipid bilayers onto only the complementary regions of uncoated glass. FIG. 1B illustrates a resultant micropatterned surface containing a grid-like array of fibronectin lines each measuring 5 μm in width and spaced 40 μm apart. Lipids in these protein corrals were both fluid and isolated from each other, as demonstrated by fluorescence recovery after photobleaching (FIGS. 2C and 2D). These patterns were stable for several days, and did not degrade over the entire duration

### Fibronectin barriers promote cell adhesion onto lipid bilayer surfaces

For example, pulmonary endothelial cells were utilized to examine cell adhesion onto surfaces containing two different geometries of fibronectin barriers. Cell adhesion experiments were carried out under serum-free conditions to minimize the effects of exogenous proteins. FIGS. 3A-3C illustrate the morphology of adherent cells six hours after seeding onto surfaces patterned with arrays of fibronectin squares surrounded by continuous membrane and passivated with BSA. Each pattern contains identical squares measuring 5 to 40 μm in width spaced 5 to 30 μm apart, surrounded by bilayers of egg PC supplemented with NBD-PE, which facilitates visualization of the supported membranes. In contrast to cells on unpatterned supported lipid bilayers which are rounded (FIG. 2B), adherent cells on substrates containing arrays of large (20-μm-wide), closely spaced (5 μm apart) squares of fibronectin exhibit a well spread morphology (FIG. 3A), resembling adherent cells on unpatterned, cell-adhesive surfaces. On these micropatterned surfaces, adherent cells attach to and extend large cellular processes across multiple fibronectin features, exposing the cell membrane to the intervening regions containing supported lipid bilayer (only 36 areal % of the surface in FIG. 3A contains supported lipid bilayers).

Decreasing the size of each square and/or spacing the features farther apart increases lipid bilayer coverage, potentially exposing a correspondingly larger area of the adherent cells to the supported membrane. However, these transformations reduce cell spreading, as illustrated in FIG. 3B. Cells on these surfaces elaborate multiple processes, which terminate on regions of fibronectin. Interestingly, this transition from well spread to branched morphology is directly correlated to lipid bilayer coverage, independent of either the size or spacing of squares alone; specifically, this transition was observed at a lipid bilayer coverage of 67 areal %. Increasing the lipid bilayer coverage to 75 areal % or greater resulted in a further decrease in cell spreading. Cells either elaborated only thin processes that ended on features of fibronectin (FIG. 3C) or, at lipid bilayer coverages of 84 areal % or greater, remained rounded and attached to individual squares of fibronectin. Thus, increasing lipid bilayer coverage on surfaces containing squares of fibronectin reduces cell spreading, potentially decreasing cell-supported membrane interaction.

Decreased cell spreading has also been correlated with a reduction in cell survival (see Ref. 19, entirely incorporated by reference herein.

Adherent cells on surfaces containing grid-like barriers of fibronectin exhibited a different pattern of cell spreading. Specifically, cells on surfaces containing grids of fibronectin surrounding square lipid corrals measuring 10- or 20-μm in width are well spread, completely covering individual corrals of lipids and extending processes along the fibronectin gridlines (FIG. 4A). In contrast, cells on surfaces containing lipid corrals measuring 40 μm in width elaborate long processes, but are not able to spread across entire corrals (FIG. 4B). Cell morphology is a function only of the spacing between gridlines and not of gridline width. Thus, in contrast to what was observed on squares of fibronectin, increasing the percentage of surface covered with lipid bilayer, in this case by using narrower gridlines, does not reduce cell spreading. Focussing on the surfaces containing lipid corrals of either 10 or 20 μm width, the gridline patterns of fibronectin promote cell spreading on surfaces comprised of up to 64 areal % of lipid bilayer. Reducing the width of the fibronectin gridlines could help increase this percentage without reducing cell spreading.

Importantly, adhesion of a cell over a lipid bilayer does not influence the fluid properties of the underlying supported membrane. FIG. 5A illustrates 6-hour adhesion of endothelial cells onto a surface containing a grid pattern of fibronectin surrounding 20-μm-wide corrals of lipid bilayers containing 1 mol % TR-PE in egg PC. After fixation of adherent cells, an electric field of 60 V/cm was applied parallel to the membrane surface, causing migration of the negatively charged TR-PE to the right side of each corral (FIG. 5B). The same gradient was formed in each corralled region, whether a cell was growing over the supported bilayer or not. The gradients could be released by turning off the electrophoretic field or reversed by inverting the polarity of the power supply. These results demonstrate that the mobility of lipids in a bilayer are not influenced by cell adhesion. In addition, this lateral lipid mobility suggests that the cell surface is separated from the supported membrane by more than 10 A, the likely extent of the dye headgroup of TR-PE above the membrane surface. In preliminary experiments, we found that large (approximately 40-nm diameter) beads attached to the headgroups of lipids in supported bilayers are not free to diffuse under adherent cells, suggesting an upper limit to the distance between the cell surface and supported membrane; this approach to measuring the vertical distance between the cell and supported membrane surfaces will be reported in detail in a subsequent communication.

In another embodiment of the invention, tissue cell culture vessels are prepared in accordance with this specification to provide a substantially lipid bilayer growth surface with cell anchoring regions formed from bilayer barrier regions further comprising a cell adhesion material such as fibronectin. Such an embodiment provides a means for growing adherent cells in a condition that more closely resembles natural conditions found in tissues and organs of the organisms from which such cells were initially derived.

### EXAMPLES

### Example 1

Vesicle Preparation

Stock solutions of small unilamellar vesicles (SUVs) were prepared by extruding solutions of 5 mg/ml of egg phosphatidylcholine (egg PC; Avanti Polar Lipids, Alabaster, Ala., USA) through 50-nm pore size polycarbonate membranes (Avanti) using a LiposoFast unit (Avestin, Inc., Ottowa, ON, Canada). For visualization of lipid bilayers, these vesicles were supplemented with either 1 mol % of Texas Redo® 1,2-dihexadecanoyl-sn-glycero-3-phosphoethanolamine (TR-PE; Molecular Probes, Eugene, Oreg., USA) or 2 mol % of 1-palmitoyl-2-[12-[(7-nitro-2-1,3-benzoxadiazol-4-yl)amino]dodecanoyl]-sn-glycero-3-phosphoethanolamine (NBD-PE; Avanti). Inclusion of either fluorescently-labeled lipid into the supported bilayers did not influence subsequent cellular response.

### Example 2

Surface Micropatterning

Protein-micropatterned lipid bilayer surfaces were prepared as outlined in FIG. 1. Borosilicate glass coverslips (VWR Scientific, Media, Pa., USA) were cleaned (Linbro 7X, ICN Biomedicals, Inc., Aurora, Ohio, USA), baked at 450° C. for 4 hours, then micropatterned with fibronectin by microcontact printing, as described in Ref. 16, Ref. 17, and Ref. 18, each entirely incorporated herein by reference. Polydimethylsiloxane (PDMS; Sylgard 184; Dow Corning, Midland, Mich., USA) elastomer stamps were oxidized in an air plasma (Harrick Scientific Corp., Ossining, N.Y.) for 20 seconds, then coated with 100 μg/ml of fibronectin (Sigma, St. Louis, Mo., USA) in 0.01 M phosphate buffer (pH 7.3) for 15 minutes. The stamps were dried under a stream of nitrogen, and then placed in contact with a coverslip for 15 minutes; a 40 g weight was placed on each 1×1 cm² stamp. The micropatterned coverslips were rinsed in phosphate buffer (PB, 0.01 M phosphate, 140 mM NaCl, pH 7.3), rinsed in water, and then dried in nitrogen. These substrates were incubated with SUVs of either egg PC, egg PC/TR-PE, or egg PC / NBD-PE (stock solutions diluted 1:3 in PB) for 30 seconds, then rinsed extensively with PB. In preparation for cell adhesion experiments, these micropatterned surfaces were incubated with 10 μg/ml of fatty-acid free bovine serum albumin (Boehringer Mannheim Biochemicals, Indianapolis, Ind., USA) in PB for 1 hour.

The two micropattern geometries that were examined contained a regular array of squares measuring either 5, 10, 20, or 40 μm in width and spaced either 5, 10, 15, 20, and 30 μm apart. One geometry consisted of square features of fibronectin, surrounded and separated by regions of lipid bilayer. Conversely, the second geometry consisted of a grid-like layout of fibronectin lines, surrounding and separating square corrals of lipid bilayer.

### Example 3

Substrate Analysis

Protein-micropatterned bilayer surfaces were examined using established fluorescence microscopy techniques. Fibronectin was immunochemically labeled with Texas Redo using standard techniques. Fluorescence recovery after photobleaching (FRAP) was used to demonstrate the fluidity of egg PC/NBD-PE lipid bilayers. On surfaces containing arrays of lipid corrals, an octagonal pattern was photobleached onto the prepared bilayer. Lipid mixing within each corral, but not between corrals, is evidenced by the establishment of a uniform fluorescence within each corral over time whose intensity is proportional to the area fraction of each corral that was photobleached. Lipid diffusion was measured quantitatively by photobleaching a linear edge onto unpatterned lipid bilayers of egg PC/ NBD-PE, and analyzing the time evolution of the fluorescence profile of this edge using a custom software package. Membrane fluidity also examined by incorporating a fluorescent, negatively charged phospholipid, TR-PE, into supported bilayers. An electric field of 60 V/cm was applied through the media (water) bathing this substrate, parallel to the membrane surface . Membrane fluidity was determined by observing whether the negatively-charged TRPE migrated in response to this applied field.

### Example 4

Cell culture

Cow pulmonary arterial endothelial cells (CPAE cells, CLL-209; American Tissue Culture Collection) were cultured in Dulbecco's Modified Eagle's Medium DMEM supplemented with 20% fetal bovine serum under standard cell culture conditions (humidified, 5% CO₂/95% air environment maintained at 37° C.). For cell adhesion experiments, CPAE cells were dissociated using a 0.25% trypsin solution, resuspended in DMEM supplemented with 10 μg/ml of Cell Tracker Blue (Molecular Probes), plated onto prepared substrates at an areal density of 1.1×10⁴ cells/cm², and then allowed to adhere for 6 hours under standard cell culture conditions. Adherent cells were then fixed with cold (4° C.) 4% paraformaldehyde for 10 minutes.

It is claimed:

1. A surface detector array device for adhering cells over lipid bilayer expanses, comprising:
a substrate having a surface defining a plurality of distinct bilayer-compatible surface regions separated by one or more bilayer barrier regions, wherein said bilayer-compatible surface regions and said bilayer barrier surface regions are formed of different materials, and said bilayer barrier regions further comprise a cell adhesion compatible material;
a lipid bilayer expanse stably localized on each of said bilayer-compatible surface regions;
an aqueous film interposed between each bilayer-compatible surface region and corresponding lipid bilayer expanse, wherein each lipid bilayer expanse is stably localized above each bilayer-compatible surface in the absence of covalent linkages between each lipid bilayer expanse and each bilayer-compatible surface, and separated therefrom by said aqueous film; and
a bulk aqueous phase covering the lipid bilayer expanses.

2. A method for adhering cells to a surface array of lipid bilayer expanses comprising:
providing a surface creating lipid bilayer compatible regions surrounded by bilayer barrier regions on said surface, wherein said bilayer barrier regions further comprise a cell adhesion compatible material;
covering said surface with a bulk aqueous phase, forming one or more lipid bilayer expanses above said lipid bilayer compatible regions wherein said lipid bilayer expanse is stably localized above said bilayer-compatible surface in the absence of covalent linkages between each lipid bilayer expanse and each bilayer-compatible surface, and separated therefrom by an aqueous film formed from a portion of said bulk aqueous phase; and,
adhering cells to said cell adhesion compatible material wherein said cells adhere only to said cell adhesion compatible material and not to said lipid bilayer expanse.
